(19) United States
(12) Patent Application Publication
(10) Pub. No.: US 2001/0056303 A1
(43) Pub. Date: Dec. 27, 2001

(54) Thoracic/abdominal wall prosthesis that stimulates and modulates connective tissue ingrowth, integrates within host tissue and allows mesothelial deposition, avoiding adhesions and erosion of the viscera

(76) Inventors: Juan Manuel Bellon Caneiro, De Renares(Madrid) (ES); Julia Bujan Varela, Madrid (ES); Pedro Lopez Hervas, Madrid (ES)

Correspondence Address: Richard P. Berg Esq., c/o LADAS & PARRY, Suite 2100, 5670 Wilshire Boulevard, Los Angeles, CA, 90036-5679, US

(21) Appl. No.: 09/884,272

(22) Filed: Jun. 19, 2001

(30) Foreign Application Priority Data
Jun. 20, 2000 (ES) P200001536

(51) Int. Cl. A61F 2/02

(52) U.S. Cl. 623/23.74; 623/23.76

(57) ABSTRACT

An implantable prosthesis for use in abdominal and thoracic surgery, in the form of a tini sheet. One of the sheet's surfaces is of a linear continuous, non-porous structure and is capable of sustaining the growth of mesothelial cells. This impedes adhesion fomation and fistulization of the viscera On the sheet's other surface, the material is discontinuous in structure with physical voids that stimulate and modulate fibroblast proliferation and collagen deposition, achieving the repair of the original defect by connective tissue. This side of the prosthesis is able to fuilly integrate within host tissue, Due to its lack of excess material, the risk of microorganisms settling on this side and of chronic infection is reduced.

The present invention is an implantable prosthesis designed to resolve defects of the abdominal or thoracic wall by stimulating the growth of connective tissue and integrating within this tissue on one of its surfaces. The other surface of the prosthesis gives support to mesothelial cells, avoiding adhesions and fistlas of the viscera. This prosthesis is intended for use in the area of abdominal and thoracic repair surgery.

### BACKGROUND OF THE INVENTION

In the field of surgical pathology, it is common to find defects of the muscuwoaponeurotic layers of the abdominal wall that support the internal viscera, appearing as abdominal or inguinal hernias, On other occasions, it is necessary to resection portions of the abdominal or thoracic wall due to tumors or infection, or defects may occur secondary to injuries due to diff~erent types of trauma. In such cases, it is often not possible or appropriate to close the defect by drawing in the layers and simple suturing. For many years, this problems has been solved through the use of artificial wall prostheses, which when sutured to the margins of the defect or over the defect are able to achieve the artificial reconstruction of the lost musculoaponeurotic substance, and thus maintain the abdominal or thoracic viscera in place.

Althotigh a variety of prostheses exist, in essence they may be classified as belonging to one of two large groups or types: 1, prostheses that are able to fiully integrate with surrounding tissue and 2, those that do not integrate and maintain their individuality.

The type 1 prostheses intimately integrate within host tissue and include the Marlex type prosthesis composed of thread that is woven in a knotted fashion leaving spaces to form a mesh or network, mainly arranged in one dimension. These mesh prostheses are currently made from plastic materials. The most commonly used material is polypropylene, but polyester, nylon and Teflon are also used and sometimes, although with fewer application, threads of reabsorbable material such as polyglactin Once implanted, these prostheses become surrounded by the ingrowth of fibroblasts and connective tissue among the spaces, and are eventually completely enveloped by host tissue. This type of prosthesis fwifils all the requirements of physical strength and, because of its complete integration within host tissue, defects are repaired in a permanent manner. However, this type of prosthesis has tavo drawbacks; one is that it carries a risk of sustained infection since, although the materials used are highly biocompatible, microorganisms are prone to settle among the knots in the meshwork and are difficult to eradicate. The second and much more important disadvantage, stems from the fact Mtat these prostheses may not be placed in direct contact with underlying viscera since this leads to the formation of adhesions with the viscera capable of causing occlusion, and in the case of the intestine, the loops adhere to the mesh provoking erosion and intestinal perforation and fistulas, which are difficult to treat and pose a threat to normal fuinction and even to life. The use of meshes composed of reabsorbable material does not resolve these problems, what is more, when the material is reabsorbed, the wall defect may reappear due to failed resistance and give rise to a relapse of the hernia,

The second type of prostheses, which do not intimately integrate with but rather become encapsulated by surrounding tissue, are designed to minimize the incidence of adhesion formation and visceral erosion. These are composed of layers of continuous plastic material with no spaces, although some are microporous such as silicon and expanded polytetrafluoroethylene (Goretex^{R}), These continuous sheets are sutured to the edges of the defect or such that they overlap the defect and never get to completely integrate with host tissue, instead, a capsule of tissue forms around the implant. Given their laminar nature, they do not cause erosion of the intestine, generally avoiding fistulas. However, among their drawbacks is an increased risk of chronic infection over the mesh prostheses, since the amnouit of foreign material is relatively greater given the extension and thickness of the layers. Moreover, microorganisms may seek refuge in the micropores and escape the action of leukocytes. If this occurs, the prosthesis generally needs to be excised creating a situation of poor control. The second large drawback of the use of a laminar prosthesis, is that the lack of tissue integration impedes the appropriate repair process by the connective tissue itself stich that hernial relapses are fairly frequent. A firther serious disadvantage of this ty of material is its considerably bigh cost. Lamninar prostheses have also been elaborated using reabsorbable material, but these do not offer great advantage yet show more drawbacks and are practically not employed.

Finally, in an attempt to maintain the advantages of these two main types of prosthesis and to avoid the possibility of intestinal erosion, composite prostheses have been designed by simply combining both types such as the prosthesis patented by Bard Inc., U.S. Pat. No. 5,593,441, whereby a Marlex tpe mesh is joined to a silicon or Goretex sheet using an adhesive or by stitching. This system simply adds the two already existing types of prostheses and poses a high risk of infection because of the amount of material. It is also less flexible and less easy to adapt and handle due to its relative thickness. Moreover, this composite prosthesis is of high manufacturing cost.

### SUMMARY OF THE INVENTION

To obtain a good wall prosthesis that fulfils the best conditions the following is ideally required: 1. A prosthesis that stimulates the ingrowth of surrounding connective tissue, allowing the proliferation of defense cells and fibroblasts within it to provide a collagenous connective incorporated into this neoformed tissue. 2. A rninimal amount of material, which if possible should be devoid of knots to avoid possible sites of bacterial growth leading to chronic infection. 3. Reduced thickness and good flexibility such that it may physically adapt to complex anatomical shapes. 4. The presence of a sheet that allows the deposition of mesothelial cells, natural components of the peritoneum and pleura, since this is the only way to impede the formation of devastating adhesions and intestinal fistulas. 5. An optimal use of materials, selecting those of high biocompatibility and tolerance to avoid a foreign body reaction. 6. An precise design in terms of physical strength properties to withstand the tension generated by defect in the region of the wall that the prosthesis replaces using the least amount of material possible. 7. A low manufacturing cost. With these conditions in mind, the thoracic/abdominal wall prosthesis that stimulates and modulates connective tissue ingiowth, integrates within host tissue and allows mesothelial deposition, avoiding adhesions and erosion of the viscera was designed and is the invention presented. The invention refers to a new type of wall prosthesis manufactured with the advantages of the two types of currently available prostheses in mind. The main drawbacks associated with their use have been eliminated. This could not be achieved by simply joining the two types of prosthesis. The prosthesis presented is composed of a thin biomaterial sheet . One of its surfaces is of a non-porous, continuous, linear structure that is very thin and flexible and is capable of acting as a substrate for the growth of mesothelial cells. This subsequently serves to impede adhesion formation and possible fistulization of the viscera.

The other side of the prosthesis is composed of a material of uneven 3-D structure rather than being flat like the existing meshes. This structure is achieved using threads of the material in the form of spirals or brushes, or as a foam, preferably comprised of open alveoli. This spatial arrangement leaves physical voids that induce the proliferation of fibroblasts and the deposition of collagen as a thick band which may be accordingly modified by varying the size and number of spaces between the material network This allows the reconstruction of the wall on this side of the prosthesis at the expense of connective tissue, which supplies the physical resistance to tension, aching the definitive repair of the defect in an unproved manner with respect to the currently available, flat meshes since these lack the capacity of modulating the response Also on this side, the prosthesis completely integrates with surrounding host tissue and thus becomes fiully incorporated. If it were necessary to avoid de long term persistence of the material, for example in the case of tissue undergoing growth or enhanced risk of infection, the uneven side of the prosthesis may be manufactured out of absorbable material such that the implant disappears once it has achieved its objective of stimulating and modulating the growth of connective tissue.

The prosthesis is elaborated out of a thin sheet, carefully avoiding excess redundant material to reduce the chances of bacterial contamination to the minimum thus lowering the risk of chronic infection.

The prosthesis may be manufactured out of a single material or the combination of biotolerated materials such as polyethylene, polypropylene, polyuretbane, or any biomaterial of appropriate resistance and flexibility. Absorbable materials may also be used, or a combination of a non-absorbable material on one side and a reabsorbable one on the other.

Given the choice of materials, ease of manufctue and the fact that it is unnecessary to prepare a knitted mesh, the result is a prosthesis of low cost. The thoracic/abdominal wall prosthesis that stimulates and modulates connective tissue ingrowth, integrates within host tissue and allows mesothelial deposition, avoiding adhesions and erosion of the viscera represents a technical advance.

### DESCRIPTION OF THE DRAWINGS

To facilitate the understanding of the invention's characteristics, a detailed description is provided based on a set of figures of a guiding rather tanm specific nature:

FIG. 1 shows an overall view in perspective of a prosthesis model of the present invention.

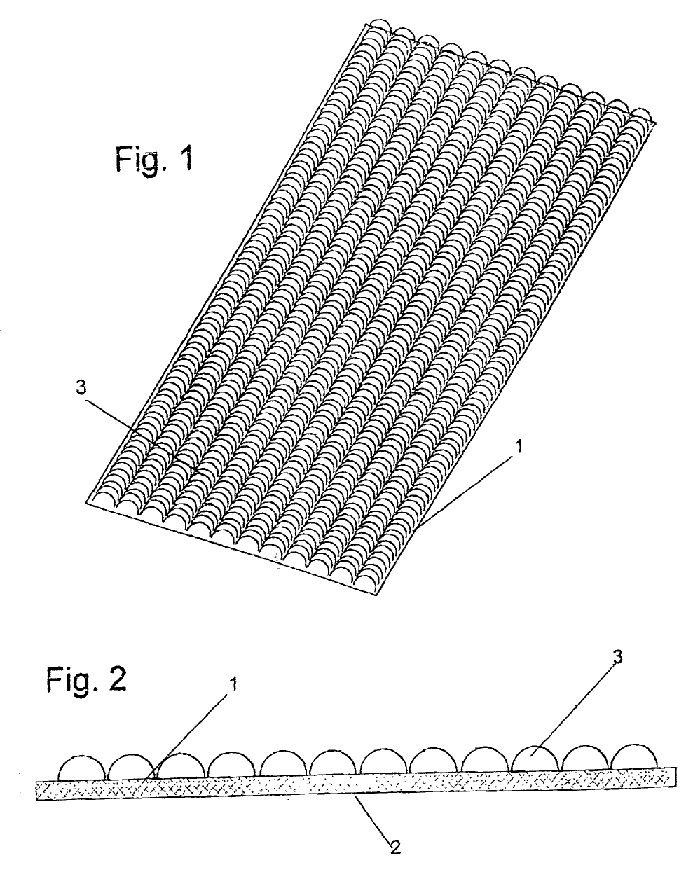

FIG. 2 represents a cross section of the model illustrated in FIG. 1.

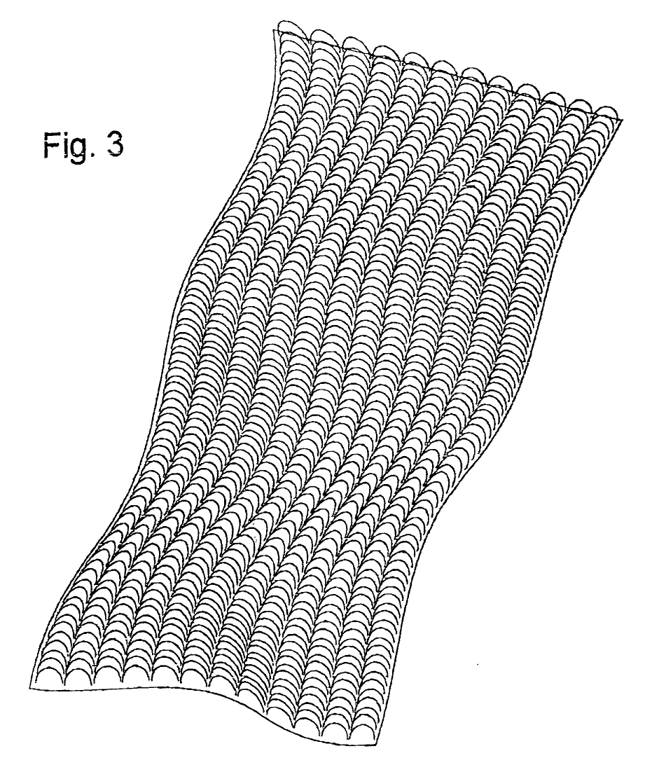

FIG. 3 shows a spatially curved view of the prosthesis model illustrated in FIG. 1. In FIGS. 4 to 7, general views and cross sections of other possible models of the present invention are provided.

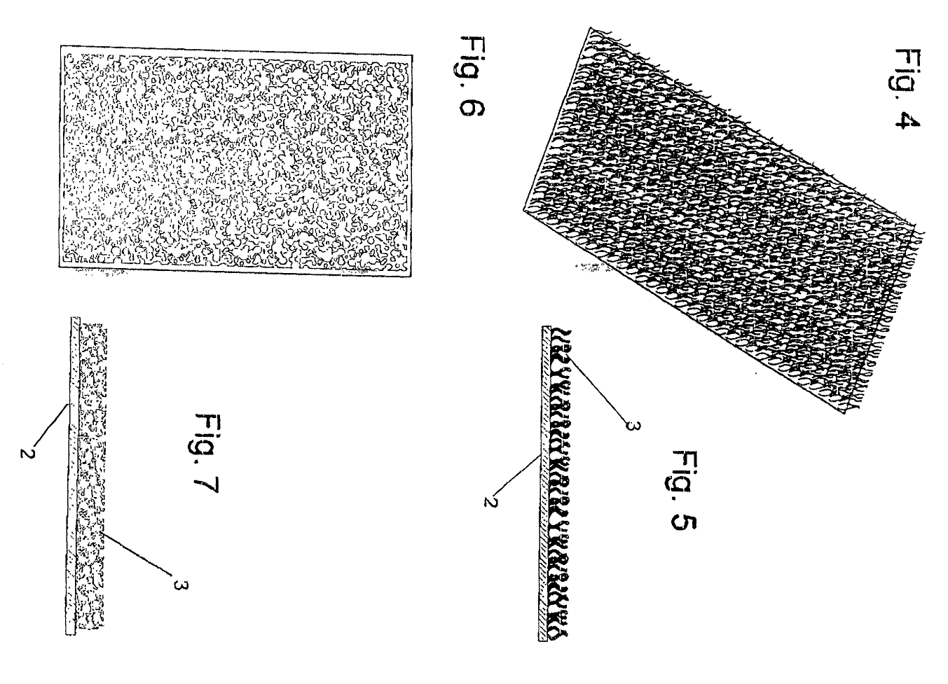

Based on the figures referred to, the prosthesis presented is comprised of a sheet of plastic (1) of resistant, flexible biotolerated material, preferably of the polyoliphin or polyirethane type.

This lamina (1) consists of a smooth surface or side (2), positioned such that it faces the peritoneal or pleural cavity. On this side, mesothelial cells constitutive of the peritoneum or pleura are able to form a natural lining, avoiding the formation of adhesions and viscera fisttlas.

On the other side, sheet 1 has a discontinuous surface and has large spaces between the material (3) which is threedimensionawly organized This is preferably achieved through the arrangement of the material in the form of a twisted fiber, brush or foam comprised of open cells or alveoli. The voids left in the material are of variable size and shape. These spaces in the material stimulate the proliferation of fibroblasts which attempt to fill them ill forming a dense layer of connective tissue that completely envelops this side of the prosthesis and in the long-term increases the structural resistance of the defect, thus avoiding hernial relapse. The thickness of the connective tissue band may be determined by varying the spaces in the discontinuous material of this side of the prosthesis, resulting in the possibility of modulating the response as required. The amount of material in the area of fibroblast stimulation and of tissue integration is the minimum needed, providing the least amount of foreign material for microorganisms to colonize, reducing the risk of infection. The material corresponding to the side of tissue proliferation and incorporation (3) may be the same as that of the main lamina (1), or of a different, even reabsorbable, material. The joining of two different materials is achieved through the use of an adhesive designed for internal use or by molding or heat sealing.

The resultant prosthesis may be easily cut to the size of the defect and may be secured to the defect's edges by suturing, or left unsutured as appropriate. The prosthesis is easy to handle due to its flexibility and malleability, and adapts perfectly to the anatomy. It may also be folded and introduced via a laparascopic trocar in this type of repair surgery. This prosthesis also has the advantages of low cost and ease of manufacture.

1. The thoracic/abdominal wall prosthesis that stimulates and modulates connective tissue ingrowth, integrates within host tissue and allows mesothelial deposition, avoiding adhesions and erosion of the viscera is essentially characterized by being composed of a sheet of biotolerated, resistant and flexible plastic, one side of which is smooth and continuous in structure with no spaces, allowing mesothelial deposition to avoid adhesions, and the other is formed by an uneven surface with three-dimensional spaces that stimulate fibroblast proliferation and tissue ingrowth.

2. The thoracic/abdominal wall prosthesis that stimulates and modulates connective tissue ingrowth, integrates within host tissue and allows mesothelial deposition, avoiding adhesions and erosion of the viscera according to claim 1, presents a discontinuous surface composed of biotolerated material spatially arranged in the form of a twisted thread, brush, or foam containing open bubbles of material. This gives rise to small 3D spaces tat vary in size and shape and stimulate the growth of fibroblasts and the laying down of collagen fibers, achieving the integration of this side of the prosthesis with host tissue.

3. The thoracic/abdominal wall prosthesis that stimulates and modulates connective tissue ingrowth, integrates within host tissue and allows mesothelial deposition, avoiding adhesions and erosion of the viscera according to claim 2, is also characterized by the fact that the three dimensional arrangement of the spaces of the discontinuous surface achieves stimulation of the connective tissue response, creating a thick band which is ideal for the permanent repair of the hernial defect.

4. The thoracic/abdominal wall prosthesis that stimulates and modulates connective tissue ingrowth, integrates within host tissue and allows mesothelial deposition, avoiding adhesions and erosion of the viscera according to claims 2 and 3, is also characterized by the fact that the size and number of voids left in the material of the discontinuous side of the prosthesis may be modified, thus allowing modulation of the connective tissue response, achieving greater or lesser thickness and greater or lesser neoformed connective tissue band width, as required in each case.

5. The thoracic/abdominal wall prosthesis that stimulates and modulates connective tissue ingrowth, integrates within host tissue and allows mesothelial deposition, avoiding adhesions and erosion of the viscera according to claims 2, 3 and 4, is also characterized by the fact that the discontinuous surface becomes enveloped by neoformed tissue achieving the complete incorporation of the prosthesis on this side.

6. The thoracic/abdominal wall prosthesis that stimulates and modulates connective tissue ingrowth, integrates within host tissue and allows mesothelal deposition, avoiding adhesions and erosion of the viscera according to claims 2, 3, 4 and 5, is also characterized by the fact that the material of the discontinuous side of the prosthesis may be reabsorbable after a period of time, once the connective tissue response has been stimulated and modulated, when it is preferable that the foreign material does not remain in the implant recipient.

7. The tlioracic/abdominal wall prosthesis that stimulates and modulates connective tissue ingrowth, integrates within host tissue and allows mesothelial deposition, avoiding adhesions and erosion of the viscera according to the previous claims, is also characterized by the fact that both sides of the prosthesis may be manufactured from the same material, of different types of material or of a combination or reabsorbable and nonreabsorbable material. Joining two types of material is achieved through molding, heat sealing or the use of an adhesive.

8. The thoracic/abdorinal wall prosthesis that stimulates and modulates connective tissue ingrowth, integrates within host tissue and allows mesothelial deposition, avoiding adhesions and erosion of the viscera according to the previous claims, is also characterized by the fact that the prosthesis is manufactured avoiding the use of supeffluous material such that sufficient resistance to tension is achieved, and the risk of infection kept to a minimum.

9. The thoracic/abdominal wall prosthesis that stimulates and modulates connective tissue ingrowth, integrates within host tissue and allows mesothelial deposition, avoiding adhesions and erosion of the viscera according to the previous claims, is also characterized by the fact tht it may be cut and sutured in its final position.

10. The thoracic/abdominal wall prosthesis that stimulates and modulates connective tissue ingrowth, integrates within host tissue and allows mesothelial deposition, avoiding adhesions and erosion of the viscera according to the previous claims, is also characterized by the fact that it is highly flexible such that it may easily be adapted to the shape of the implant site or be folded and positioned through a laparoscopy trocar.
